(19) United States
(12) Patent Application Publication
(10) Pub. No.: US 2024/0041511 A1
(43) Pub. Date: Feb. 8, 2024

(54) ENERGY GENERATOR WITH MULTIFUNCTIONAL ENERGY RECEPTACLE

(71) Applicant: Covidien LP, Mansfield, MA (US)

(72) Inventors: Tom E. McMunigal, Mead, CO (US); Lewis R. Puterbaugh, Longmont, CO (US); Jeffrey A. Hammond, Longmont, CO (US); Travis A. Jones, Erie, CO (US)

(73) Assignee: Covidien LP, Mansfield, MA (US)

(21) Appl. No.: 18/265,714

(22) PCT Filed: Dec. 14, 2021
(86) PCT No.: PCT/US2021/063193

§ 371 Date: Jun. 7, 2023

Related U.S. Application Data
(60) Provisional application No. 63/124,968, filed on Dec. 14, 2020.

(51) Int. Cl. A61B 18/12

(52) U.S. Cl. CPC A61B 18/1206; A61B 2018/00994

(57) ABSTRACT

An energy generator includes a plurality of energy sources. Each energy source includes: a power supply configured to output a direct current waveform; an inverter coupled to the power supply and configured to generate an electrosurgical waveform or an ultrasonic drive waveform; and an energy source controller configured to control the inverter and the power supply. The generator also includes a plurality of receptacles each of which is coupled to the plurality of energy sources. Each receptacle includes a plurality of ports, where a first portion of the ports is configured to transmit the electrosurgical waveform and a second portion of the ports is configured to transmit the ultrasonic drive waveform.

### CROSS-REFERENCE TO RELATED APPLICATION

The present application claims priority to U.S. Provisional Application No. 63/124,968, filed on Dec. 14, 2020.

### BACKGROUND

### Technical Field

The present disclosure relates to a surgical energy delivery system. In particular, the present disclosure relates to an energy generator configured to operate in a plurality of energy delivery modes. The energy generator includes one or more multifunctional receptacles configured to interface with a variety of energy delivery devices.

### Background of Related Art

Ultrasonic and electrosurgical devices are frequently used during surgical procedures to limit bleeding and to minimize injury to tissue. Ultrasonic surgical devices and systems utilize ultrasonic energy, i.e., ultrasonic vibrations, to treat tissue. More specifically, ultrasonic surgical devices and systems utilize mechanical vibration energy transmitted at ultrasonic frequencies to coagulate, cauterize, fuse, seal, cut, and/or desiccate tissue to effect hemostasis. An ultrasonic surgical device may include, for example, an ultrasonic blade and a clamp mechanism to enable clamping of tissue against the blade. Ultrasonic energy transmitted to the blade causes the blade to vibrate at very high frequencies, which heats tissue clamped against or otherwise in contact with the blade.

Electrosurgery involves application of high radio frequency electrical current to a surgical site to cut, ablate, desiccate, or coagulate tissue. In monopolar electrosurgery, a source or active electrode delivers radio frequency alternating current from the energy generator to the targeted tissue. A patient return electrode is placed remotely from the active electrode to conduct the current back to the generator.

In bipolar electrosurgery, return and active electrodes are placed in close proximity to each other such that an electrical circuit is formed between the two electrodes (e.g., in the case of an electrosurgical forceps). In this manner, the applied electrical current is limited to the tissue positioned between the electrodes. Accordingly, bipolar electrosurgery generally involves the use of devices where it is desired to achieve a focused delivery of electrosurgical energy between two electrodes.

Currently, separate generators are used to energize ultrasonic and electrosurgical devices. Moreover, simultaneous activation of electrosurgical devices also requires the use of corresponding number of energy generators. Accordingly, there is a need for an energy generator configured to interface with a variety of ultrasonic and electrosurgical devices.

### SUMMARY

The present disclosure provides a surgical energy delivery system including an energy generator having one or more multifunctional receptacles configured to couple to a variety of energy (i.e., ultrasonic and electrosurgical) devices. This allows for replacement of multiple plug designs which require multiple receptacles and/or adapters with a uniform plug and receptacle. The energy generator is configured to detect a type of the energy device that is coupled to the multifunctional port. The multifunctional receptacle includes multiple portions of contacts, each of the portions configured to energize a specific type of energy devices (e.g., ultrasonic, monopolar, bipolar, etc.). Upon detection of the type of the energy device, the energy generator configures its output and the multifunctional receptacle to energize the energy device in one of available energy delivery modes. The energy generator may be configured to output energy on two separate channels generated by two separate energy sources. Each of the sources may include a power supply configured to output DC power and an alternating current power inverter configured to output an RF waveform or an ultrasonic drive signal for energizing a transducer of an ultrasonic instrument.

According to one embodiment of the present disclosure, an energy generator is disclosed. The energy generator includes a plurality of energy sources. Each energy source includes: a power supply configured to output a direct current waveform; an inverter coupled to the power supply and configured to generate an electrosurgical waveform or an ultrasonic drive waveform; and an energy source controller configured to control the inverter and the power supply. The generator also includes a plurality of receptacles each of which is coupled to the plurality of energy sources. Each receptacle includes a plurality of ports, where a first portion of the plurality of ports is configured to transmit the electrosurgical waveform and a second portion of the plurality of ports is configured to transmit the ultrasonic drive waveform.

Implementations of the foregoing embodiment may include one or more of the following features. According to one aspect of the above embodiment, the energy generator may include: a main controller may be configured to make a determination of a type of an instrument coupled to at least one receptacle of the plurality of receptacles. The main controller may be further configured to instruct the energy source controller to output either the electrosurgical waveform or the ultrasonic drive waveform based on the determination. The main controller may be further configured to activate at least one of the first portion or the second portion of the ports based on the determination. Each receptacle includes a plurality of contacts. Each port of the plurality of ports includes at least one contact of the plurality of contacts. Each port defines a pair of longitudinal surfaces. Each port may also include a portion of the plurality of contacts disposed along each longitudinal surface of the pair of longitudinal surfaces.

According to another embodiment of the present disclosure, a surgical energy delivery system is disclosed, and includes an energy generator. The generator includes: a plurality of energy sources, each energy source of includes: a power supply configured to output a direct current waveform; an inverter coupled to the power supply and configured to generate an electrosurgical waveform or an ultrasonic drive waveform; and an energy source controller configured to control the inverter and the power supply. The generator also includes a plurality of receptacles each of which is coupled to the plurality of energy sources, each receptacle of the plurality of receptacles including a plurality of ports, where a first portion of the ports is configured to transmit the electrosurgical waveform and a second portion of the ports is configured to transmit the ultrasonic drive waveform. The system also includes a plurality of energy delivery instruments, each energy delivery instrument includes a plug configured to couple to one receptacle of the plurality of receptacle.

Implementations may include one or more of the following features. According to one aspect of the above embodiment, the energy generator further includes a main controller configured to make a determination of a type of an energy delivery instrument of the plurality of energy delivery instruments coupled to at least one receptacle of the plurality of receptacles. The main controller is further configured to instruct the energy source controller to output either the electrosurgical waveform or the ultrasonic drive waveform based on the determination. The main controller is further configured to activate at least one of the first portion or the second portion of the ports based on the determination. Each receptacle includes a plurality of receptacle contacts. Each port includes at least one contact of the plurality of receptacle contacts. Each port defines a pair of longitudinal surfaces. Each port includes a portion of the plurality of receptacle contacts disposed along each longitudinal surface of the pair of longitudinal surfaces. The plug includes a substrate having a plurality of extensions. The plurality of extensions is configured to be inserted into the plurality of ports. Each extension includes a first surface having at least one first plug contact and a second surface having at least one second plug contact. The substrate may be a printed circuit board and the at least one first plug contact and the at least one second plug contact may be conductive traces disposed on the printed circuit board.

### BRIEF DESCRIPTION OF THE DRAWINGS

The present disclosure may be understood by reference to the accompanying drawings, when considered in conjunction with the subsequent, detailed description, in which:

FIG. 1 is a perspective view of a surgical energy delivery system according to an embodiment of the present disclosure;

FIG. 2 is a front view of an energy generator of FIG. 1 according to an embodiment of the present disclosure;

FIG. 3 is a schematic diagram of the energy generator of FIG. 1 according to an embodiment of the present disclosure;

FIG. 4 is a perspective view of a plug according to an embodiment of the present disclosure;

FIG. 5 is a perspective view of a receptacle for receiving the plug according to an embodiment of the present disclosure; and

FIG. 6 is a perspective view of a connector of the receptacle of FIG. 5.

### DETAILED DESCRIPTION

Embodiments of the presently disclosed surgical energy delivery system are described in detail with reference to the drawings, in which like reference numerals designate identical or corresponding elements in each of the several views. As used herein the term “distal” refers to the portion of the surgical device coupled thereto that is closer to the patient, while the term “proximal” refers to the portion that is farther from the patient.

The term “application” may include a computer program designed to perform functions, tasks, or activities for the benefit of a user. Application may refer to, for example, software running locally or remotely, as a standalone program or in a web browser, or other software which would be understood by one skilled in the art to be an application. An application may run on a controller, or on a user device, including, for example, a mobile device, an IOT device, a server system, or any programmable logic device.

In the following description, well-known functions or constructions are not described in detail to avoid obscuring the present disclosure in unnecessary detail. Those skilled in the art will understand that the present disclosure may be adapted for use with either an endoscopic device, a laparoscopic device, or an open device. It should also be appreciated that different electrical and mechanical connections and other considerations may apply to each particular type of device.

An energy generator according to the present disclosure may be used in ultrasonic or electrosurgical (i.e., monopolar and/or bipolar) procedures, including, for example, cutting, coagulation, ablation, and vessel sealing procedures. The generator may include a plurality of outputs for interfacing with various ultrasonic and electrosurgical devices (e.g., ultrasonic dissectors and hemostats, monopolar devices, return electrode pads, bipolar electrosurgical forceps, footswitches, etc.). Further, the generator may include electronic circuitry configured to generate radio frequency energy specifically suited for powering ultrasonic devices and electrosurgical devices operating in various electrosurgical modes (e.g., cut, blend, coagulate, division with hemostasis, fulgurate, spray, etc.) and procedures (e.g., monopolar, bipolar, vessel sealing).

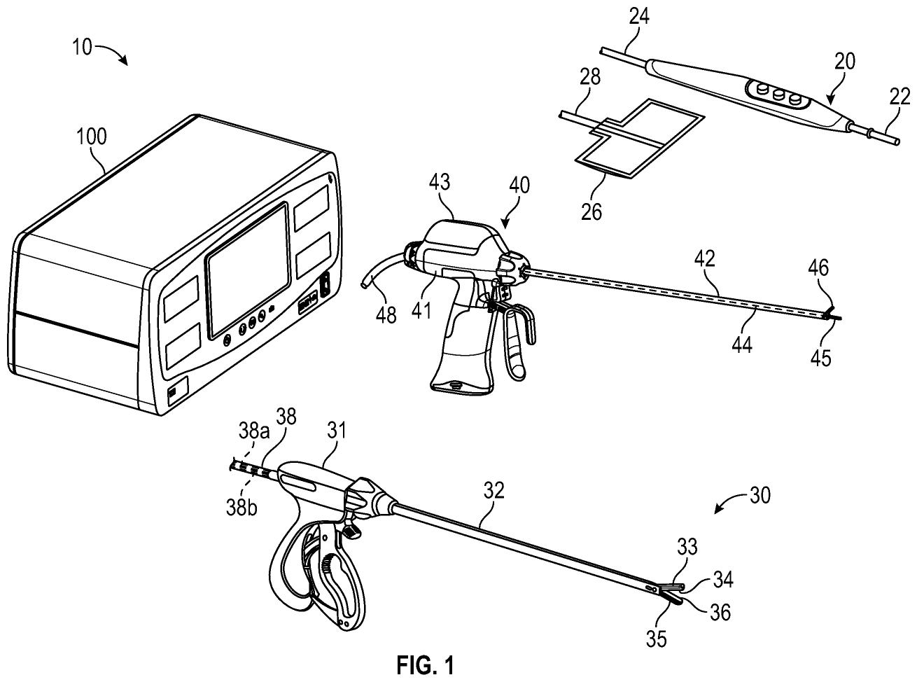

Referring to FIG. 1 a surgical energy delivery system 10 includes an energy generator 100 which may be used with one or more monopolar electrosurgical instruments 20, one or more bipolar electrosurgical instruments 30, one or more ultrasonic instruments 40, and/or any other suitable energy delivery instrument. The monopolar electrosurgical instrument 20 includes an active electrode 22 (e.g., electrosurgical cutting probe, ablation electrode(s), etc.) for treating tissue of a patient. The system 10 may include a plurality of return electrode pads 26 that, in use, are disposed on a patient to minimize the chances of tissue damage by maximizing the overall contact area with the patient. The return electrode pad 26 is electrically coupled to the generator 100 via a supply line 28. Electrosurgical alternating RF waveform is supplied to the instruments 20 by the generator 100 via supply line 24.

The bipolar electrosurgical instrument 30 may be forceps or tweezers. The bipolar electrosurgical instrument 30 includes a housing 31 and a pair of opposing jaw members 33 and 35 disposed at a distal end of a shaft 32 coupled to the housing 31. The jaw members 33 and 35 have one or more active electrodes 34 and a return electrode 36 disposed therein, respectively. The active electrode 34 and the return electrode 36 are connected to the generator 100 through cable 38 that includes the supply and return lines 38a, and 38b.

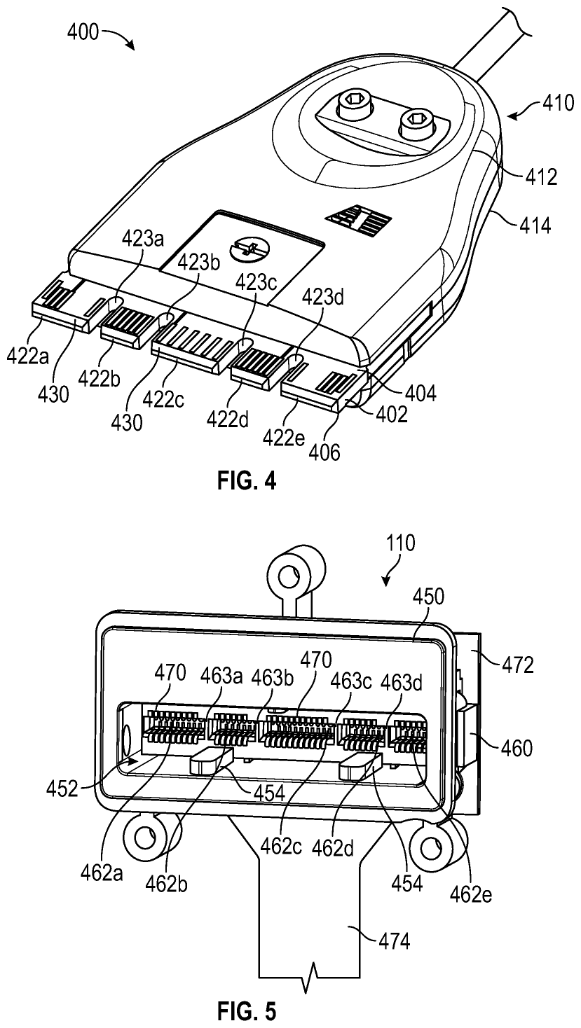

The ultrasonic instrument 40 includes a housing 41 and a shaft 42 extending distally from the housing 41. An ultrasonic transducer 43 is coupled to the housing 41 and is coupled to a waveguide 44. A blade 45 is defined at a distal end of the waveguide 44 and a jaw member 46 is pivotally coupled to the shaft 42 allowing for clamping of tissue against the blade 45. The transducer 43 is configured to convert electrical energy into ultrasonic vibrations transmitted along the waveguide 44 to the blade 45. The ultrasonic instrument 40 also includes a cable 48 for connection to the generator 100. Each of the instruments 20, 30, and 40 includes a plug 400 (FIG. 4) for coupling to the generator 100.

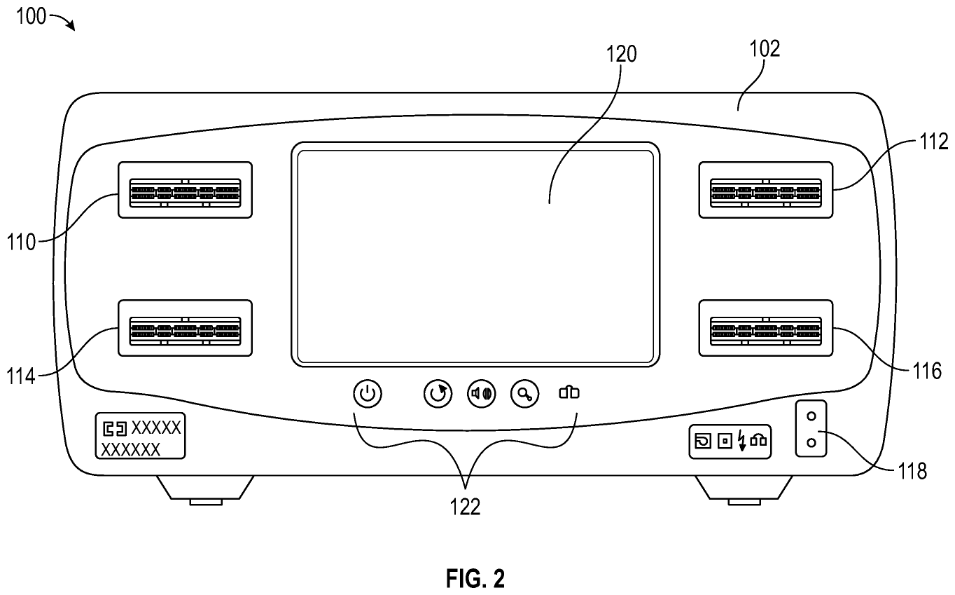

With reference to FIG. 2, a front face 102 of the generator 100 is shown. The generator 100 may include a plurality of receptacles 110, 112, 114, 116 each of which is configured to couple to various types of energy instruments (i.e., instruments 20, 30, 40) and a receptacle 118 for coupling to the return electrode pad 26. The generator 100 includes a display 120 for providing the user with variety of output information (e.g., intensity settings, treatment complete indicators, etc.). The display 120 is a touchscreen configured to display a corresponding menu for the devices being used. The user then adjusts inputs by simply touching corresponding menu options. The generator 100 also includes suitable input controls 122 (e.g., buttons, activators, switches, touch screen, etc.) for controlling the generator 100.

The generator 100 is configured to operate in a variety of modes, which includes outputting electrosurgical or ultrasonic waveforms based on the selected mode. Each of the electrosurgical modes output electrosurgical waveforms based on a preprogrammed power curve that dictates how much power is output by the generator 100 at varying impedance ranges of the load (e.g., tissue). Each of the power curves may also include power, voltage, and current control ranges that are defined by the user-selected intensity setting and the measured impedance of the load. In ultrasonic mode, the generator 100 outputs an ultrasonic drive signal, which is an alternating current waveform suitable for energizing the transducer 43 of the ultrasonic instrument 40.

The electrosurgical waveforms are radio frequency waveforms, which may be either continuous or discontinuous and may have a carrier frequency from about 200 kHz to about 500 kHz. As used herein, continuous waveforms are waveforms that have a 100% duty cycle. In embodiments, continuous waveforms are used to impart a cutting effect on tissue as well as soft coagulation, bipolar, and vessel seal. Conversely, discontinuous waveforms are waveforms that have a non-continuous duty cycle, e.g., below 100%. In embodiments, discontinuous waveforms are used to provide coagulation effects to tissue. The ultrasonic drive signal is continuous and may have a carrier frequency from about 20 kHz to about 60 kHz.

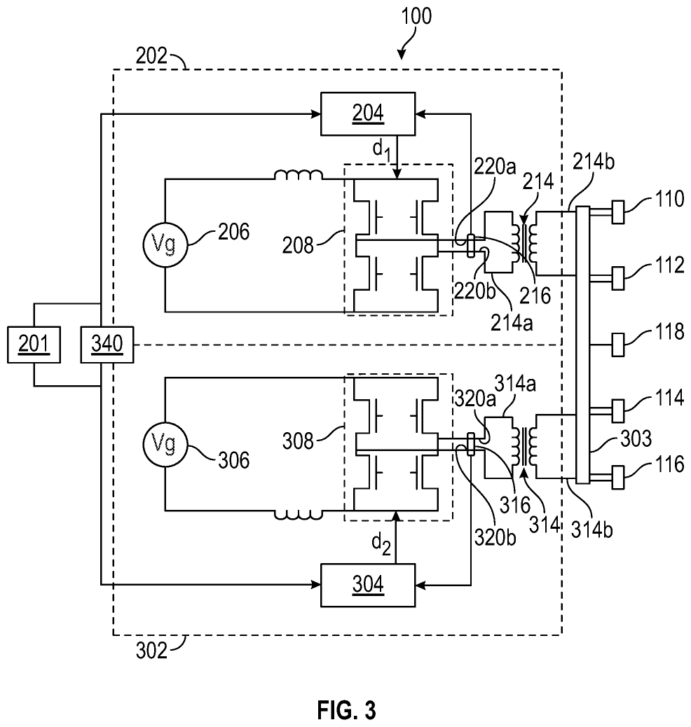

With reference to FIG. 3, the generator 100 may have a multiple energy source architecture, where each energy source is supplied by an individual and separate inverter, each of which is powered by an individual and separate DC power supply. More specifically, the generator 100 includes a first energy source 202 and a second energy source 302. Each of the sources 202 and 302 includes a first controller 204 and a second controller 304, a first power supply 206 and a second power supply 306, and a first inverter 208 and a second inverter 308. The power supplies 206 and 306 may be high voltage, DC power supplies connected to a common AC source (e.g., line voltage) and provide high voltage, DC power to their respective inverters 208 and 308, which then convert DC power into a first and second RF waveforms or ultrasonic drive signals.

The receptacles 110, 112, 114, 116, 118 are coupled to the sources 202 and 302 through a switching relay 303, which enables pathways for energizing connected instruments 20, 30, 40. The switching relay 303 may include a plurality of high frequency switching components, e.g., MOSFETS, etc. When the monopolar electrosurgical instrument 20 is connected to one of the receptacles 110, 112, 114, or 116, the receptacle 118 is also connected to the one of the energy sources 202 or 302 to enable the return electrode pad 26. In embodiments, the generator 100 may operate with two monopolar electrosurgical instruments 20 sharing a common return electrode pad 26. Two monopolar electrosurgical instruments 20 may be activated simultaneously, each being energized by a corresponding energy source 202 or 302. In this embodiment, both of the sources 202 and 302 are connected to the receptacle 118 allowing for a common return path. In embodiments, the receptacles 110 and 112 may be energized by the first source 202 and the receptacles 114 and 116 may be energized by the second energy source 302. In further embodiments, plurality of other instruments, i.e., bipolar instruments 30 and ultrasonic instruments 40, may be used simultaneously and in any suitable combination, i.e., matching or mismatching pairs.

The switching relays 303 are coupled to the inverter 208 through an isolation transformer 214. The isolation transformer 214 includes a primary winding 214a coupled to the inverter 208 and a secondary winding 214b coupled to the switching relays 303. Similarly, the switching relays 303 are coupled to the inverter 308 through an isolation transformer 314. The isolation transformer 314 includes a primary winding 314a coupled to the inverter 308 and a secondary winding 314b coupled to the switching relays 303.

The inverters 208 and 308 are configured to operate in a plurality of modes, during which the generator 100 outputs corresponding waveforms having specific duty cycles, peak voltages, crest factors, etc. It is envisioned that in other embodiments, the generator 100 may be based on other types of suitable power supply topologies. inverters 208 and 308 may be resonant RF amplifiers or non-resonant RF amplifiers, as shown. A non-resonant RF amplifier, as used herein, denotes an amplifier lacking any tuning components, i.e., inductors, capacitors, etc., disposed between the inverter and the load, e.g., tissue.

The generator 100 also includes a main controller 201, which is responsible for operation of the generator 100 including user input and output, configuration of the first and second energy sources 202 and 302, as well as configuration of the receptacles 110, 112, 114, 116, 118. The controllers 201, 204, 304 may include a processor (not shown) operably connected to a memory (not shown), which may include one or more of volatile, non-volatile, magnetic, optical, or electrical media, such as read-only memory (ROM), random access memory (RAM), electrically-erasable programmable ROM (EEPROM), non-volatile RAM (NVRAM), or flash memory. The processor may be any suitable processor (e.g., control circuit) adapted to perform the operations, calculations, and/or set of instructions described in the present disclosure including, but not limited to, a hardware processor, a field programmable gate array (FPGA), a digital signal processor (DSP), a central processing unit (CPU), a microprocessor, and combinations thereof. Those skilled in the art will appreciate that the processor may be substituted for by using any logic processor (e.g., control circuit) adapted to perform the calculations and/or set of instructions described herein.

Each of the controllers 204 and 304 is operably connected to the respective power supplies 206 and 306 and/or inverters 208 and 308 allowing the processor to control the output of the first energy source 202 and the second source 302 of the generator 100 according to either open and/or closed control loop schemes. A closed loop control scheme is a feedback control loop, in which a plurality of sensors measures a variety of tissue and energy properties (e.g., tissue impedance, tissue temperature, output power, current and/or voltage, etc.), and provide feedback to each of the controllers 204 and 304. The controllers 204 and 304 then control their respective power supplies 206 and 306 and/or inverters 208 and 308, which adjust the DC and/or RF waveform, respectively.

The generator 100 according to the present disclosure may also include a plurality of sensors 216 and 316, each of which monitors output of the first energy source 202 and the second energy source 302 of the generator 100. The sensors 216 and 316 may be any suitable voltage, current, power, and impedance sensors. In the embodiment illustrated in FIG. 3, the sensors 216 are coupled to leads 220a and 220b of the inverter 208. The leads 220a and 220b couple the inverter 208 to the primary winding 214a of the transformer 214. The sensors 316 are coupled to leads 320a and 320b of the inverter 308. The leads 320a and 320b couple the inverter 308 to the primary winding 314a of the transformer 314. Thus, the sensors 216 and 316 are configured to sense voltage, current, and other electrical properties of energy being supplied. In embodiments, the sensors 216 and 216 may sense energy properties at the secondary windings 214b and 314b.

In further embodiments, the sensors 216 and 316 may be coupled to the power supplies 206 and 306 and may be configured to sense properties of DC current supplied to the inverters 208 and 308. The controllers 204 and 304 also receive input signals from the display 120 and the input controls 122 of the generator 100 and/or controls of the instruments 20, 30, 40. The controllers 204 and 304 adjust power outputted by the generator 100 and/or perform other control functions thereon in response to the input signals.

The inverters 208 and 308 include a plurality of switching elements, which may be arranged in an H-bridge topology. In embodiments, inverters 208 and 308 may be configured according to any suitable topology including, but not limited to, half-bridge, full-bridge, push-pull, and the like. Suitable switching elements include voltage-controlled devices such as transistors, field-effect transistors (FETs), combinations thereof, and the like. In embodiments, the FETs may be formed from gallium nitride, aluminum nitride, boron nitride, silicone carbide, or any other suitable wide bandgap materials.

The controllers 204 and 304 are in communication with the respective inverters 208 and 308. Controllers 204 and 304 are configured to output control signals, which may be pulse-width modulated (“PWM”) signals. In particular, controller 204 is configured to modulate a control signal d1 supplied to switching elements of the inverter 208 and the controller 304 is configured to modulate a control signal d2 supplied to switching elements of inverter 308. The control signals d1 and d2 provide PWM signals that operate the inverters 208 and 308 at their respective selected carrier frequency. Additionally, controller 204 and 304 are configured to calculate power characteristics of output of the first energy source 202 and the second source 302 of the generator 100, and control the output of the first energy source 202 and the second source 302 based at least in part on the measured power characteristics including, but not limited to, voltage, current, and power at the output of inverters 208 and 308.

Each of the controllers 204 and 304 is coupled to a clock source 340, which acts as a common frequency source for each of the controllers 204 and 304, such that the controllers 204 and 304 are synced. The clock source 340 may be an electronic oscillator circuit that produces a clock signal for synchronizing operation of the controllers 204 and 304. In particular, sampling operation of the controllers 204 and 304 may be synchronized. Each of the controllers 204 and 304 generates a waveform based on clock signal from the clock source 340 and the selected mode. Thus, once the user selects one of the electrosurgical modes or ultrasonic modes, each of the controllers 204 and 304 outputs a first and second control signal, which are used to control the respective inverters 208 and 308 to output first and second RF waveforms corresponding to the selected mode. The selected mode for each of the first energy source 202 and the second source 302, and the corresponding RF waveforms, may be the same or different.

With reference to FIG. 4, a universal plug 400, shown as a male connector, is coupled to each of the instruments 20, 30, and 40. The plug 400 includes a substrate 402 having a first surface 404 and a second surface 406 that is on the opposite side of the first surface 404. The substrate 402 is enclosed in a housing 410 having a first shell 412 and a second shell 414, which may be coupled using any suitable method, such as fasteners, adhesives, ultrasonic welding, etc. The substrate 402 may be a multilayer printed circuit board (PCB) formed from any suitable dielectric material, including, but not limited to, composite materials composed of woven fiberglass cloth with an epoxy resin binder such as FR-4. The substrate 402 includes an insertion portion 420 having a plurality of extensions 422a-e separated by a plurality of cutouts 423a-d. In embodiments, the insertion portion 420 may include any number (n) of extensions separated by corresponding number (n−1) of cutouts. In further embodiments, the insertion 420 may be continuous without any cutouts.

Each of the extensions 422a-e includes a plurality of plug contacts 430 disposed on both surfaces 404 and 406. The contacts 430 may be conductive traces formed on the surfaces 404 and 406. Each of the contacts 430 are isolated from each other and some or all are coupled to the components of the instrument, i.e., instrument 20, 30, or 40. Each of the extensions 422a-e may include any number of contacts, which may be from 1 to 6 contacts 430 on each of the surfaces 404 and 406, providing for a total of 10 to 60 contacts. A group of contacts 430 may be used to transmit a single signal.

In embodiments, the contacts 430 of the extension 422a may be used by the generator 100 to interrogate the instruments 20, 30, 40, i.e., to determine a type of the instrument, read number of uses, load button configurations, and energy delivery profiles. In particular, the instruments 20, 30, 40 may include a storage device (not shown) storing various data corresponding to the instrument that is accessible by the generator 100 through the contacts 430 of the extension 422a. Similarly, the generator 100 is configured to write data to the storage device, i.e., usage data, time stamps, etc. In embodiments, various feedback from the instruments 20, 30, 40, such as sensor signals and operational parameters may also be transmitted through the contacts 430 of the extension 422a.

The contacts 430 of the extension 422e may be used for receiving control signals form the instruments 20, 30, 40. In embodiments, the instruments 20, 30, 40 may include controls, e.g., buttons or triggers, used for activating and deactivating energy application. The control signals are outputted in response to engagement of the instrument controls.

The contacts 430 of the extension 422b and 422d may be used for transmission of electrosurgical waveforms to the monopolar instrument 20 and the bipolar instrument 30. In embodiments, the contacts 430 of the extension 422b are coupled to the active electrode 22 of the monopolar instrument 20 and the active electrode 34 of the bipolar instrument 30. The contacts 430 of the extension 422d are coupled to the return electrode 36. With respect to the monopolar instrument 20, the extension 422d is not used, since the return electrode pad 26 provides for a return path through the receptacle 118. The contacts 430 of the extension 422c may be used for transmission of the ultrasonic drive signal for energizing the ultrasonic instrument 40.

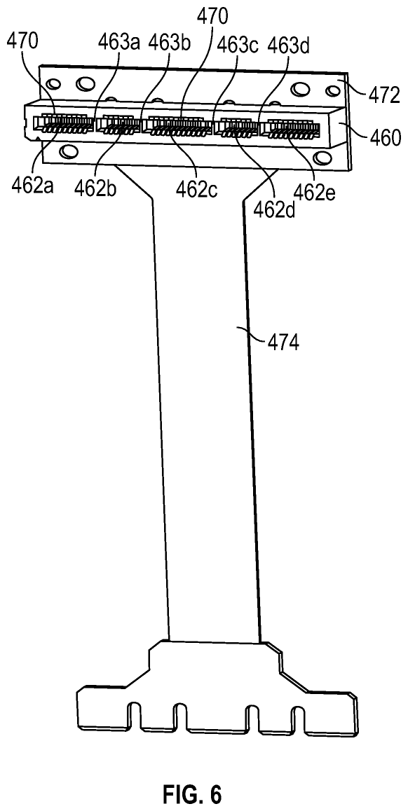

With reference to FIGS. 5 and 6, the receptacle 110, which is identical to the receptacles 112, 114, and 116, includes a cover 450 having an opening 452. The cover 450 also includes one or more protrusions 454 configured to engage the housing 410 of the plug 400, such that the plug 400 is properly oriented relative to the receptacle 110 preventing improper insertion. The receptacle 110 includes a connector 460 disposed within the opening 452. The connector 460 includes a plurality of ports 462a-e separated by a plurality of partitions 463a-d. The ports 462a-e may have a rectangular, slit-like shape configure configured to receive the corresponding extensions 422a-e such that the partitions 463a-d also engage, i.e., fit within, the respective cutouts 423a-d.

The receptacle 110 also includes a plurality of receptacle contacts 470. Each of the ports 462a-e includes one or more contacts 470 along both longitudinal surfaces of the ports 462a-e. Each of the ports 462a-e may include any number of contacts, which may be from 1 to 6 contacts 470 on each of the longitudinal surfaces providing for a total of 10 to 60 contacts. The receptacle contacts 470 are configured to engage the plug contacts 430. The connector 460 may be disposed on a substrate 472 for mounting the receptacle 110 to a housing of the generator 100. The connector 460 is coupled to the switching relay 303 via a flexible cable 474. The switching relay 303, along with other components of the generator 100 may be disposed on a mother board PCB having an edge connector, which is coupled to the flexible cable 474.

The generator 100 according to the present allows the receptacles 110, 112, 114, 116 to be used with any of the instruments 20, 30, 40 by utilizing a single receptacle and plug design. The generator 100 operates the receptacles 110, 112, 114, 116 by initially determining the type of the instrument that is connected thereto through the port 462a, which receives the extension 422a of the plug 400. The generator 100 interrogates the connected instrument to determine whether it is one of the monopolar instrument 20, the bipolar instrument 30, or the ultrasonic instrument 40. Based on the determination, the generator 100 enables or disables the ports 462b-d. For the monopolar instrument 20, the generator 100 enables only the port 462b, which couples to the extension 422b of the plug 400 for transmission of electrosurgical energy to the monopolar instrument 20. For the bipolar instrument 30, the generator 100 enables the ports 462b and 462d, which couple to the extensions 422b and 422d of the plug 400. For the ultrasonic instrument 40, the generator 100 enables the port 462c, which couples to the extension 422c of the plug 400. The port 462e may be enabled by the generator 100 in response to the presence of sensors, or other devices disposed in the instruments 20, 30, 40 designed to provide status, measurements, or other feedback. Thus, in the absence of sensors the port 462e may be disabled. In addition to configuring the receptacles 110, 112, 114, 116 the generator 100 also configures the first and second energy sources 202 and 302 to deliver the requested waveforms to energize the connected instruments. In particular, the main controller 201 instructs the first controller 204 and/or the second controller 304 to output a requested waveform based on the coupled instrument and user selections. While the present disclosure described the plug 400 as being a male plug and the receptacles 110, 112, 114, 116 as being female, it is envisioned that these configurations may be reversed.

While several embodiments of the disclosure have been shown in the drawings and/or described herein, it is not intended that the disclosure be limited thereto, as it is intended that the disclosure be as broad in scope as the art will allow and that the specification be read likewise. Therefore, the above description should not be construed as limiting, but merely as exemplifications of particular embodiments. Those skilled in the art will envision other modifications within the scope of the claims appended hereto.

What is claimed is:

1. An energy generator comprising:
a plurality of energy sources, each energy source of the plurality of energy sources includes:
a power supply configured to output a direct current waveform;
an inverter coupled to the power supply and configured to generate an electrosurgical waveform or an ultrasonic drive waveform; and
an energy source controller configured to control the inverter and the power supply; and
a plurality of receptacles each of which is coupled to the plurality of energy sources, each receptacle of the plurality of receptacles including a plurality of ports, wherein a first portion of the plurality of ports is configured to transmit the electrosurgical waveform and a second portion of the plurality of ports is configured to transmit the ultrasonic drive waveform.

2. The energy generator according to claim 1, further comprising:
a main controller configured to make a determination of a type of an instrument coupled to at least one receptacle of the plurality of receptacles.

3. The energy generator according to claim 2, wherein the main controller is further configured to instruct the energy source controller to output either the electrosurgical waveform or the ultrasonic drive waveform based on the determination.

4. The energy generator according to claim 2, wherein the main controller is further configured to activate at least one of the first portion or the second portion of the ports based on the determination.

5. The energy generator according to claim 1, wherein each receptacle of the plurality of receptacles includes a plurality of contacts.

6. The energy generator according to claim 5, wherein each port of the plurality of ports includes at least one contact of the plurality of contacts.

7. The energy generator according to claim 6, wherein each port of the plurality of ports defines a pair of longitudinal surfaces.

8. The energy generator according to claim 7, wherein each port of the plurality of ports includes a portion of the plurality of contacts disposed along each longitudinal surface of the pair of longitudinal surfaces.

9. A surgical energy delivery system comprising:
an energy generator including:
a plurality of energy sources, each energy source of the plurality of energy sources includes:
a power supply configured to output a direct current waveform;
an inverter coupled to the power supply and configured to generate an electrosurgical waveform or an ultrasonic drive waveform; and
an energy source controller configured to control the inverter and the power supply; and
a plurality of receptacles each of which is coupled to the plurality of energy sources, each receptacle of the plurality of receptacles including a plurality of ports, wherein a first portion of the ports is configured to transmit the electrosurgical waveform and a second portion of the ports is configured to transmit the ultrasonic drive waveform; and
a plurality of energy delivery instruments, each energy delivery instrument of the plurality of energy delivery instruments includes a plug configured to couple to one receptacle of the plurality of receptacle.

10. The surgical energy delivery system according to claim 9, wherein the energy generator further includes a main controller configured to make a determination of a type of an energy delivery instrument of the plurality of energy delivery instruments coupled to at least one receptacle of the plurality of receptacles.

11. The surgical energy delivery system according to claim 10, wherein the main controller is further configured to instruct the energy source controller to output either the electrosurgical waveform or the ultrasonic drive waveform based on the determination.

12. The surgical energy delivery system according to claim 11, wherein the main controller is further configured to activate at least one of the first portion or the second portion of the ports based on the determination.

13. The surgical energy delivery system according to claim 9, wherein each receptacle of the plurality of receptacles includes a plurality of receptacle contacts.

14. The surgical energy delivery system according to claim 13, wherein each port of the plurality of ports includes at least one contact of the plurality of receptacle contacts.

15. The surgical energy delivery system according to claim 14, wherein each port of the plurality of ports defines a pair of longitudinal surfaces.

16. The surgical energy delivery system according to claim 15, wherein each port of the plurality of ports includes a portion of the plurality of receptacle contacts disposed along each longitudinal surface of the pair of longitudinal surfaces.

17. The surgical energy delivery system according to claim 9, wherein the plug includes a substrate having a plurality of extensions.

18. The surgical energy delivery system according to claim 17, wherein the plurality of extensions is configured to be inserted into the plurality of ports.

19. The surgical energy delivery system according to claim 17, wherein each extension of the plurality of extensions includes a first surface having at least one first plug contact and a second surface having at least one second plug contact.

20. The surgical energy delivery system according to claim 19, wherein the substrate is a printed circuit board and the at least one first plug contact and the at least one second plug contact are conductive traces disposed on the printed circuit board.
